(19) United States
(12) Patent Application Publication
(10) Pub. No.: US 2004/0193096 A1
(43) Pub. Date: Sep. 30, 2004

(54) Conceptus chamber

(76) Inventor: William Isaac Cooper, Tulsa, OK (US)

Correspondence Address: WILLIAM ISAAC COOPER, 9224 SOUTH LAKEWOOD, TULSA, OK, 74137, US

(21) Appl. No.: 10/396,526

(22) Filed: Mar. 25, 2003

(51) Int. Cl. A61N 1/30

(52) U.S. Cl. 604/19

(57) ABSTRACT

The Conceptus Chamber is an enclosed sterile chamber designed to support and maintain a conceptus until maturity. A novel fluid system of nutrition, novel methods of fetal evaluation, and a novel oxygenation device and systems are described. New surgical and therapeutic methods connected with use of the Conceptus Chamber are taught. Novel methods of utilization of the Conceptus Chamber with nonhuman and nonplacental species are taught. A dedicated, integrated cart for use with the Conceptus Chamber is described.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCES TO SEQUENCE LISTING, A TABLE, OR A COMPUTER PROGRAM LISTING COMPACT DISK APPENDIX

Not Applicable.

### BACKGROUND

1. Field of the Invention

The primary background of the invention is found in two of my prior inventions: U.S. Pat. No. 5,218,958, was filed Feb. 21, 1991 in field 128/205.26, and was titled Placental Chamber—Artificial Uterus; the second, U.S. Pat. No. 6,001,552, was filed in July, 1998 in field 435/1.1, and was titled Method for Supporting the Life of a Premature Baby.

2. Discussion of Prior Art

One of the most heart-breaking areas of medicine is the care of newborns at the threshold of viability, as discussed in the American College of Obstetrics Practice Bulletin Number 38: at the lowest end of current treatment options, prognosis is so grim that it is often chosen to simply withhold treatment and allow the baby to expire.

The primary objective of this and those prior inventions is to salvage the life of a fetus or severely premature baby which cannot or, for medical reasons, should not remain in the womb until the time of viability, which is defined as that point in development when the baby can survive outside the womb. Salvaging is accomplished by transfer and maintenance of the fetus in an environment which adequately recapitulates the intrauterine environment. The present invention addresses problems with the above inventions, and reveals the development of significant changes, advances and improvements on the prior art, as well as novel devices, methods and uses.

Indications for use of this invention can arise for various medical reasons. Maternal indications could include sudden, imminent or threatened demise of the mother; maternal diseases, such as diabetes mellitus or hypertension; indications for chemotherapy or radiation therapy to the mother, which might damage the fetus; indications for surgical procedures to the mother, which might damage the fetus; and maternal cardiac or cardiovascular disease.

Indications associated with the gestation directly could include uterine rupture or dehiscence, ectopic pregnancy, placental abruption or placental abnormalities, hypertensive disorders of pregnancy, diabetes of pregnancy, heart failure in pregnancy, or any other medical disorder caused or aggravated by pregnancy.

Indications associated with the fetus or conceptus could include fetal anomalies or problems requiring medical or surgical care of the fetus which must or should be performed on the fetus outside of the womb; chorioamnionitis; failure to thrive; or evidence of anoxia or fetal distress.

Use of the Conceptus Chamber for any of the above indications would be at the discretion of the practitioner(s) and patient, with fully informed consent whenever possible.

Secondary objectives are aimed at novel uses associated with veterinary and agricultural applications. Veterinary obstetrical indications would be similar to those in the human. Veterinary economic indications would include utilizing the present invention to increase the number of offspring, and safety of increased numbers of fetuses, per gestation. Other needs and uses would include the preservation and propagation of endangered species, where environmental pollutants have caused endangered oocyte-laying species to suffer reproductive incapacity by inadequate calcification, or where attempts at reproduction of endangered species in zoological parks have been unproductive. This brief list would not preclude other medical, economic and ecological indications.

Research and development of the two above-mentioned patents and consideration of the problems which the present invention addresses have brought to light several new ideas where significant changes and improvements can be made, as well as new devices, methods and uses, which constitute the ideas and claims presented in the present application.

The term fetus, where used throughout this patent application and for purposes of this invention, is intended to include a fetus or multiple fetuses in any stage of development. The term conceptus refers to the entire products of conception, which include the fetus, umbilical cord, placenta and chorioamnion; the latter is also commonly referred to as the membranes or bag of waters. For purposes of this invention the term conceptus may also refer to the above definition although the chorioamnion has been opened and the amniotic fluid lost, in which case it would imply the fetus and placenta with the intact umbilical cord connecting them; this latter case is also called the fetoplacental unit. Finally, for the purpose of utilization of this invention for nonplacental species, the term conceptus would refer to the fetus and as much of the other products of conception as would be available, as well as any accompanying covering or shell.

### BRIEF SUMMARY OF THE INVENTION

The Conceptus Chamber is an enclosed sterile chamber designed to support and maintain a conceptus until maturity. A novel fluid system of nutrition, novel methods of fetal evaluation, and a novel oxygenation device and systems are described. New surgical and therapeutic methods connected with use of the Conceptus Chamber are taught. Novel methods of utilization of the Conceptus Chamber with nonhuman and nonplacental species are taught. A dedicated, integrated cart for use with the Conceptus Chamber is described.

### BRIEF DESCRIPTION OF THE DRAWINGS

FIG. 1-A: the Conceptus Chamber and conceptus in operation.

The walls of the Conceptus Chamber (1) and its supporting bottom define a single chamber open at the top and covered with a protective lid (2). There are four openings through the walls of the chamber: an inlet (3) for oxygen with or without other gases, an outlet (4) for oxygen and/or other gases, an inlet (5) for fluids and an outlet (6) for fluids. The exact placement of these may vary, depending on which will provide the optimal maintenance of the conceptus. The fluid inlet/outlet system may be used to replace, to alter, or simply to agitate the fluid within the chamber.

The conceptus (7) is resting in a supportive net (8) which itself is attached to a firm supportive ring (9). The supportive ring has two handles (10) for handling, removing and replacement of the net; it is held in place by resting on bolsters or supports (11) on the side walls of the chamber.

The fluid (12), within which the conceptus is maintained, fills the chamber to a fluid level (13) sufficient to cover the conceptus. At the same time, the fluid level is low enough to allow an upper level of gases (14) above the fluid, and to allow outflow of gases at the gas outlet (4).

The gas inlet (3) houses a tube open to a key element of the invention, the Conceptus Chamber Oxygenation Device (15) which, with the gas under controlled pressure as it enters the inlet, will expel bubbles of oxygen-rich gas (16) directly into the fluid via small openings (17) through the device, the size and placement of which are optimal for oxygenation of the conceptus, and especially the placenta. The gas will thus bubble up to and around the conceptus on a continual basis for as long as the conceptus remains in the chamber, at a rate and in a manner optimal to the oxygenation of the conceptus, and especially the placental surface. A second benefit of this bubbling process would be the continual agitation of the fluid around the conceptus, facilitating not only oxygen transfer but also transfer of nutrients to, and removal of metabolic byproducts from, the conceptus.

A temperature-controlling device (18) with controllable tolerances is incorporated; here, the temperature-controlling device lies directly beneath the chamber, although it could be made to surround the chamber more completely if preferable.

An echogenic fetal heart rate monitor transducer is shown mounted on the wall (19) of the chamber. Alternatively, said fetal heart rate monitor transducer could be placed within the chamber. On the net is a pulse oximeter (20) which measures the oxygenation of the blood in the placenta.

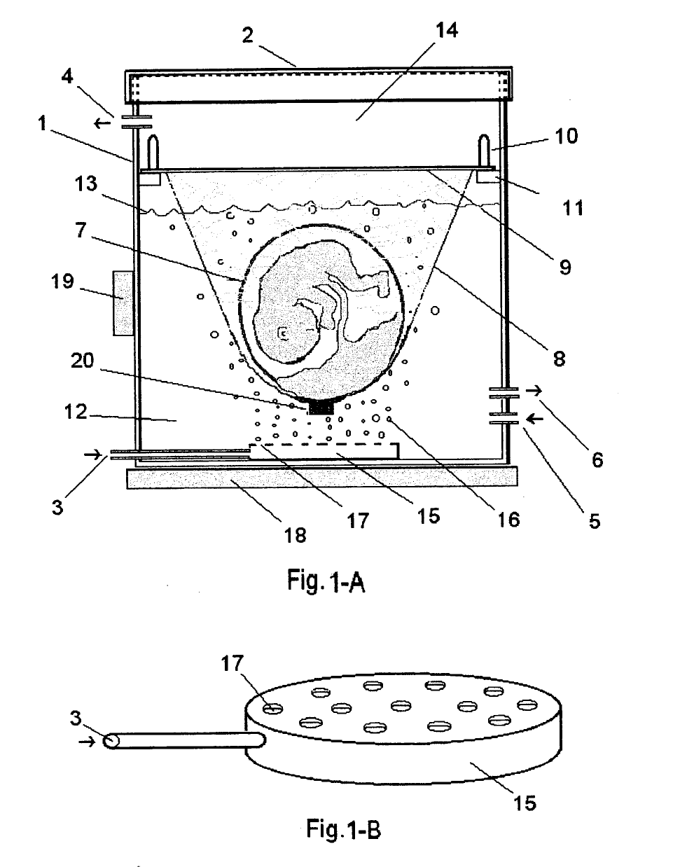

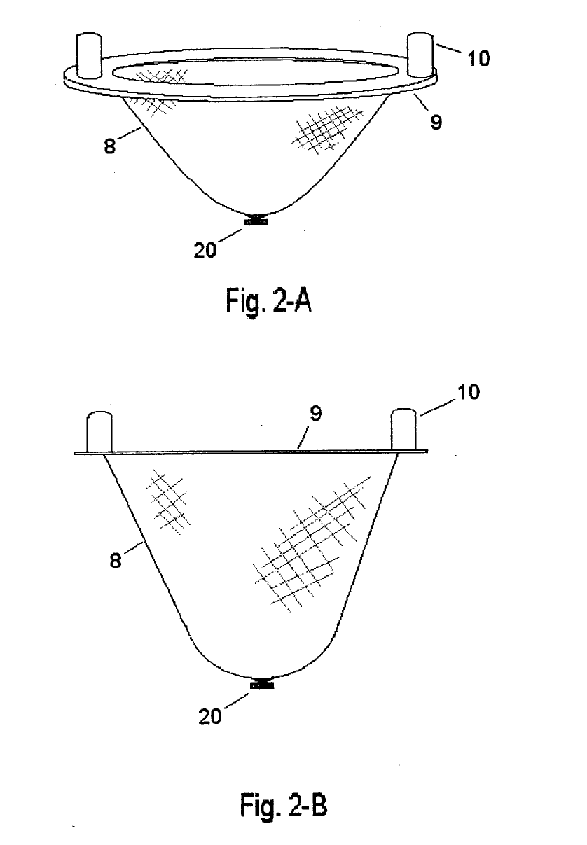

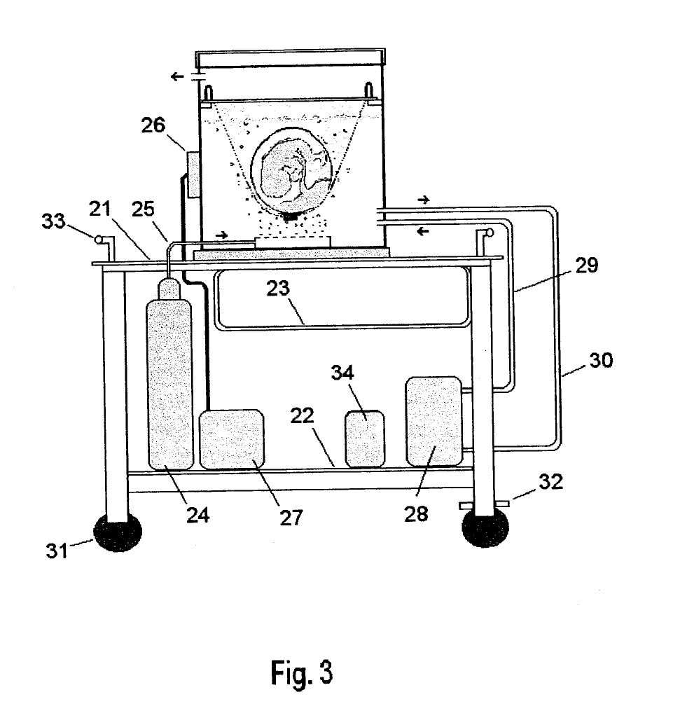

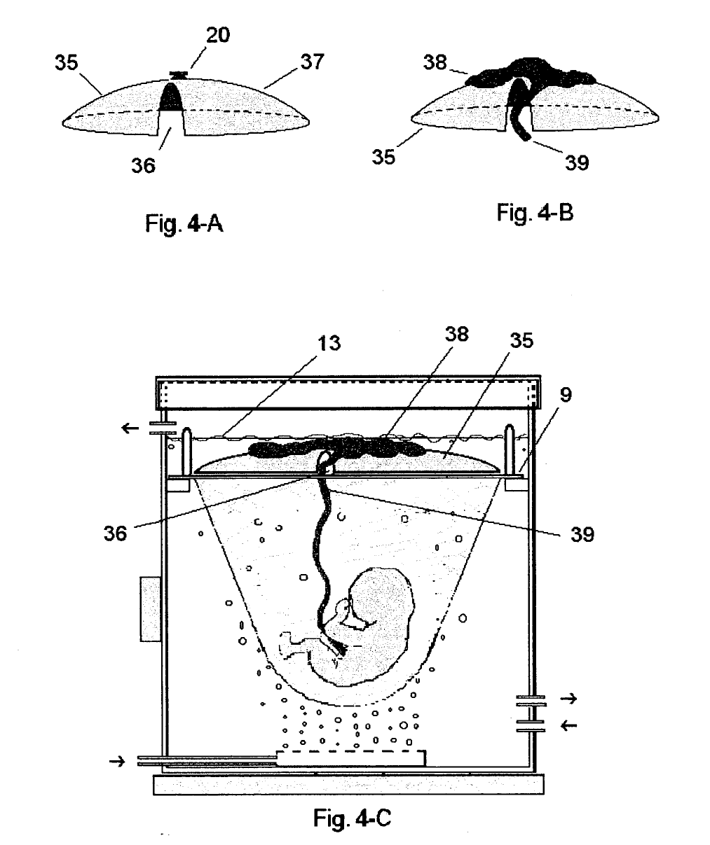

FIG. 1-B: the Oxygenation Device:

shows an angled view of the Conceptus Chamber Oxygenator, which has no moving parts: the inlet (3) passes oxygen-rich gas into the simple chamber which has openings at the top whence the bubbles exit.

FIG. 2-A: the conceptus support net viewed at a slight angle to show the opening of the support ring through which the conceptus is passed.

The net (8) which houses the conceptus has openings large enough to permit optimal contact between the bubbles and fluid and the conceptus. It is fixed to a flat, firm support ring (9) which in turn rests on supports on the walls of the Conceptus Chamber. The handles (10) are located on either upper side of the support ring.

FIG. 2-B: the conceptus support net viewed horizontally.

FIG. 3: the Conceptus Chamber cart.

The Conception Chamber Cart is self-contained, with easily sterilized top (21) surface, bottom (22) surface, and utility shelf (23) surface. Firmly secured are the tank (24) for oxygen or oxygen-rich gases, which is connected by a tube (25) to the gas inlet; the fetal heart rate monitor transducer (26) is shown connected to the housing for its monitor and the pulse oximeter monitor (27), including alarms for each. The electrical pump for the fluid circulation (28) is connected by tubing to the fluid inlet (29) and outlet (30) openings of the Conceptus Chamber. The wheels (31) are six inch or greater in diameter to minimize jarring, and at least one set of wheels is locking (32). Handles at each end of the cart (33) assist control during transport. There is a battery (34) on the bottom shelf.

FIG. 4-A: The Placental Support dome.

The convex Placental Support dome (35) has a bottom diameter slightly smaller than the diameter of the support ring holding the net housing the fetus, and when in place sits securely upon the support ring holding the net. It has on one side a slotted opening (36) which is wide enough for the umbilical cord to go through during placement of the placenta and fetus in the Conceptus Chamber, and to remain there without compression by the weight of the placenta.

FIG. 4-B: The Placental Support Dome supporting the placenta.

The placenta (38) is placed and remains atop the dome (37) of the Placental Support in such a way that the umbilical cord (39) remains attached to both the placenta and the fetus.

FIG. 4-C the Conceptus Chamber in the case where the Placental Support dome is used.

The dome (35) rests on the ring support for the net (9). The placenta (37) is resting on top of the Placental Support dome, and the attached umbilical cord (38) passes through the opening in the Placental Support (35) on its way to the attached fetus. The fluid level (13) has been raised to cover the placenta.

### DETAILED DESCRIPTION OF THE INVENTION

The invention is a life support system for a premature fetus. Throughout this specification, any reference to a fetus is to be considered either singular or plural, since this invention is applicable to either single or multiple pregnancies. The term conceptus refers to the entire products of a conception, including the fetus, umbilicus, placenta, amniotic fluid and chorioamnion; for purposes of this specification the term conceptus can also refer to more than one conceptus. This specification also deals with cases in which the chorioamnion has been ruptured and the amniotic fluid lost, in which cases the term conceptus would refer to the remaining constituents of the conceptus, namely, the fetus and placenta with their connecting umbilical cord.

The uses of this invention would include salvage of a fetus which is determined to have no anomalies incompatible with life.

Several, but not all possible indications have been addressed in Background of the Invention: they include dangers to the fetus, to the mother, or to both. In veterinary, agricultural and ecological applications, indications would be determined by the practitioners and owners or overseers of the animals.

Concerning the Walls, Internal Surfaces, Top and Net of the Conceptus Chamber:

The walls of the chamber are partially or completely transparent to permit continual visual observation of the conceptus; opacity would be provided as needed to permit normal ocular development and prevention of light-associated fetal damage. The walls of the chamber would be made of material compatible with use of various ultrasound and radiological methods of intra-chamber fetal evaluation.

There are openings through the wall for inflow of gas, outflow of gas, inflow of fluid and outflow of fluid. These openings are connect respectively to the inflow hose from the gas source, the outflow hole for gas, the inflow tubing for fluid and the outflow tubing for fluid; the latter two may be connected to a recirculating pump.

Some or all of the internal surfaces of the invention, including the walls of the Conceptus Chamber, the net, and other surfaces within the chamber or tubing associated with the chamber, may have antimicrobial coatings as taught by Jaimiolsky et alius in U.S. Pat. No. 6,514,517, filed June, 2001; or by Zhong in U.S. Pat. No. 6,468,649, filed in December, 1999; or in one or a combination of antimicrobial coatings as might be developed in the future. Alternatively, or in addition, part or all of the Conceptus Chamber could be constructed of compositions containing antimicrobial substances, as taught by Albers et alius in U.S. Pat. No. 6,455,059, filed in May, 2001. This is a novel use for these coatings and substances: prior art only describes their use limited to small medical devices placed within the body.

The top of the chamber is of dimensions adequate to permit easy removal and closure, while preventing contamination of the interior of the chamber when the top is in place. A safety mechanism may be placed whereby the top cannot be inadvertently opened.

The net of the Conceptus Chamber consists of a material which is strong enough to hold the conceptus up to and including the projected size of the conceptus at the expected time of removal from the chamber. It is flexible enough to permit normal fetal flexion and movement, with resistance adequate to aid the fetus in normal musculoskeletal development.

Concerning Multiple Concepti:

Special variations in technique might be required in multiple gestation: delivery of all concepti together and intact might be required to prevent disruption of the amniotic sacs. Such requirement may be determined by ultrasound prior to delivery. In such a case, a Conceptus Chamber is chosen with dimensions optimal to accommodate the entirety of the concepti, which may thereby be delivered into the Conceptus Chamber as one unit. In veterinary medicine, this might prove the standard method for litters. In such cases, multiple pulse oximeters and/or fetal heart rate monitors could monitor each fetus individually. Alternatively, if the chorioamnions are discreet enough to separate without tearing during delivery, multiple Conceptus Chambers might be available at the operative site for individual care of each conceptus, as in the case of human dichorionic twins.

Should the chamber be used in the alternative method described below for cases with the chorioamnion being ruptured, the net would be of material whose openings are small enough to prevent fetal parts from passing through the net.

The net is supported by a firm ring, which in turn rests on supporting structures on the sides of the chamber, at a level which will keep the conceptus below the surface of the fluid within the chamber. The net support ring has handles by which it may be removed from, and replaced into, the chamber without touching the net or conceptus.

Concerning Nonplacental Species:

Another variation in the net would involve use of the Conceptus Chamber to allow continued development of nonplacental species, including oocyte-laying species: in such a case, the conceptus could be supported and held together in a properly sized net or other support structure to maintain its integrity until such time the fetus would normally hatch, as closely as can be determined by the practitioner. In such a case the Conceptus Chamber might need to provide no more than homeostatic temperature and an aseptic environment which mimics that which is normal for development for that particular species, and resistance to fetal movement equivalent to that which would be provided by a healthy, normal shell.

In cases of nonplacental species the net would thus be of parameters consistent with the normal fetal environment for that species at the given stage of fetal development, and may more closely resemble a shell.

Concerning the Dedicated Cart for the Conceptus Chamber:

A dedicated mobile cart is described, embodying easily sterilized surfaces, including those of the upper and lower surfaces and utility shelf surface; the cart has handles, and locking wheels large enough to prevent jarring of the Conceptus Chamber during transport. There are auxiliary devices connected to the Conceptus Chamber which may be incorporated into the mobile cart: the gas tank for controlled passage of gas into the Oxygenation Device; the fluid pump for controlled delivery of fluid into the chamber; the temperature control device; the fetal heart rate monitor and fetal pulse oximeter monitor; the battery; and alarm devices to alert practitioners to inappropriate function or readings in any of the above.

The parameters of the cart may be adapted to various uses: in-hospital, ambulance, farm or agricultural uses in which it may be transported in a pick-up truck, or even adaptations for uses in the most remote areas.

Concerning the Fluid to be Used Within the Conceptus Chamber:

The fluid contains a physiologic, balanced, aqueous solution and suspension of all requisite nutrients for development of the conceptus; it is present within the chamber during use.

The fluid inlet and outlet system may be capped or closed when not in use; there may be rubber coverings through which hypodermic needles could be passed for placement and/or removal of fluids as needed. Alternatively, there could be continuous or intermittent flow via this system.

The fluid may also be used as a vehicle for introduction and use of antimicrobial and antibiotic agents into the chamber and to the conceptus for prophylaxis or treatment of sepsis, infection and microbial contamination.

The outflow fluid may be evaluated for monitoring microbial presence in the chamber or for determining efficacy of antibiotic and antimicrobial prophylaxis or treatment; the fluid may be used for monitoring amounts and uptake of various nutrients by the conceptus, as well as metabolic by-products of the conceptus; this information can be used in treatment and nutritional management decisions. Should antibiotic or antimicrobial therapy be required or ongoing, it may be advisable to transfer the conceptus into a new, sterile chamber; this could be performed with a new net or the same net.

Concerning the Method of Oxygenation of the Conceptus:

The unique Conceptus Oxygenation Device incorporated in this invention delivers oxygen-rich gas directly into the fluid within the chamber, the flow being controlled passively by the number, size and placement of the holes in the wall of the Oxygenation device and controlled actively by the controlled and monitored pressure of the gas administered, as well as by the percentage of oxygen in the gas. The gas may contain pure oxygen or a specific mixture of oxygen and other gases. This allows direct oxygenation of the conceptus by gaseous oxygen acting directly at the level of the villi of the placenta.

Alternatively, if the parameters of the placenta require such oxygenating agents in the fluid, this invention makes new uses of recently patented, superior oxygenation vehicles such as ultrapure hemoglobin solution as taught by Rausch et alius in U.S. Pat. No. 6,506,725, filing date May, 1999; mutant hemoglobins as taught by Segall et alius in U.S. Pat. No. 6,486,123, filing date May, 2000; or one of a large number of fluorocarbon liquids as taught by Faithfull et alius in U.S. Pat. No. 6,289,892, filing date March, 2000. These might be necessary, for example, when a placenta has adequate thickness to prevent bubbles of oxygen to reach the deeper villous surfaces: in such case, the present invention teaches a novel use of those vehicles as well as a novel method of such use and a novel composition containing such vehicles.

Concerning the Surgical Method:

When it is determined that the circumstances of a pregnancy make it a candidate for use of this invention, the pregnancy is scheduled to be delivered by a novel method of obstetrical delivery.

First, the pregnancy is evaluated by ultrasound and any other pertinent parameters to evaluate the gestation completely, with emphasis on stage of development; number and placement of conceptus or concepti; in cases of multiple pregnancy whether the concepti, whether each have a separate chorioamnion; the thickness of the uterine wall at the anticipated incision site; the amount of amniotic fluid; and location of placenta or placentae in order to assure the placental dissection being the final step in the removal of the intact conceptus from the uterus. Also determined is the absence of any contraindications to surgery, such as unexpected fetal demise or fetal anomalies incompatible with ultimate survival.

Pre-operative preparation of the chamber: prior to incision, and under sterile conditions, the Conceptus Chamber is prepared: the Conceptus Chamber and fluid delivery/recovery system is filled with the appropriate nutrient fluid to a level sufficient to cover the candidate conceptus in the support net. Next, oxygen flow is begun through the oxygenation device at the bottom of the chamber, and will continue for as long as the fetus remains in the chamber. The entire unit, including fluid, is brought to an optimal, controlled temperature prior to and during delivery, and thereafter as long as the fetus resides in the Conceptus Chamber.

Preparation of the patient: prior to surgery, thorough ultrasound assessment of the pregnancy has confirmed the parameters of the pregnancy, and lack of contraindications to surgery. The operating room is laid out in such a way as to keep the Conceptus Chamber within the sterile surgical field prior to and during delivery of the conceptus into the chamber.

During surgery, the Conceptus Chamber and attendants thereto stand adjacent to the surgical team, properly gowned and gloved. Standard operative technique is used to enter the abdomen and womb, the uterine incision being guided by the underlying position of the placenta. The uterine incision is begun by a small nick traversing the thickness of the uterine wall down to but not including the underlying amniotic sac. The uterine incision is then carried lengthwise, avoiding the uterine arteries, by blunt dissection or by bandage scissors, to avoid the possibility of lacerating the amnion or the placenta. The incision is made large enough for the entire conceptus to be removed intact without restraint by the opening of the incision. During this time, all conceptus surfaces are kept moist with physiological salt solutions as needed, and no dry sponges or materials are placed in contact with the conceptus; these steps prevent abrasive or dehydration damage to the surface of the conceptus. At any point in this step of the surgical procedure, if an inadvertent breech is made in the amniotic sac it should be clamped and tied off with permanent suture tightly enough to prevent leakage of amnniotic fluid.

After the uterine incision is deemed adequate, the entire conceptus, with membranes intact, is gently separated from the uterine wall by blunt dissection; the last portion to be thus bluntly dissected is the placental surface.

Next, removal of the entire, intact conceptus is performed at once: the fetus, still connected to the placenta by the umbilical cord, remains within the amniotic fluid of the unruptured chorioamniotic sac: the surgeon places the intact conceptus into the Conceptus Chamber net which an assistant is holding, by means of the supportive ring of the net, next to the incision site; the assistant then places the net containing the conceptus securely onto the net supporting structures located on the inner walls of the Conceptus Chamber. Alternatively, the surgeon may place the conceptus directly into the net in the Conceptus Chamber, without removing the net from the chamber. Placement is done in such a way that the placental surface will be left facing toward the Oxygenation Device, so as to receive optimal oxygenation when placed in the Conceptus Chamber; and in such a way that part of the placental surface will be in contact with the pulse oximeter located in or on the net. Next, the lid is placed over the top and the Conceptus Chamber is removed from the operative field by means of its previously described cart.

The Conceptus Chamber, with oxygenation device in operation and now containing the support net holding the conceptus suspended in the fluid within the Chamber, is taken to the designated room in which it is to be maintained, while continuing oxygenation and homeostatic temperature control.

The fluid is agitated by the continuous flow of gas from the Oxygenation Device; if more vigorous or more precisely directed flow of the fluid is required, the Conceptus Chamber fluid is maintained under continuous or intermittent flow through fluid outlet and inlet ports. If more vigorous or precisely directed oxygenation is required, the oxygenation device could be made to flow over a larger surface area and from additional ports, for example in cases where the placental cotyledons are located all around the conceptus.

Further concerning the surgical method, alternative protocols are taught, which may be required in cases of chorioamnionitis, infection of the conceptus, or irreparable loss of the integrity of the chorioamnion.

In case of chorioamnionitis or infection of the conceptus, whether suspect or proven, the following method of delivery is used: upon entering the uterine cavity, samples are taken for laboratory detection, identification and analysis of potential pathogens; when delivered, the conceptus is delivered into a container of physiologic fluid containing antibiotics and antimicrobials, alone or in combination, at levels appropriate with adequate, rapid destruction of pathogens without danger to the conceptus. Within this container is an Oxygenation Device as previously described, to oxygenate the conceptus during this initial stage of antimicrobial therapy. Subsequent to this initial stage of therapy, which should last no longer than necessary to minimize the superficial septic burden of the conceptus, the appropriate antibiotic and antimicrobial agent or agents should be placed in the fluid within the Conceptus Chamber and administered by placement within the fluid in such a way as to keep therapeutic levels at all times. During this therapy the fluid will be monitored for pathogens to evaluate therapeutic response so that therapeutic agents may be increased, changed, or discontinued as indicated.

Concerning surgical technique, an alternative protocol must be followed in the case of rupture of the chorioamnion, either spontaneous or iatrogenic, which is beyond repair. In such a case the fetus must be delivered in such a way as to prevent an attempt at respiration: attempted respiration, even by a severely premature fetus, can institute physiological reactions in the fetal cardiovascular system. These reactions can change the fetal circulation, which shunts fetal blood into the umbilical cord for oxygenation and alimentation by the placenta, and shut off the umbilical blood flow in favor of pulmonary blood flow. Should this irreversible event transpire, and the fetus be too premature to oxygenate its blood through the lungs, the condition is terminal.

Therefore in such an instance the delivering physician keeps the fetal mouth covered and immediately passes the fetus into the fluid: if under aseptic conditions, the fluid of the Conceptus Chamber; if under septic conditions, the fluid of the container described above for delivery in conditions of amnionitis or sepsis. The placenta is then quickly dissected bluntly from the uterine wall and placed appropriately: in the latter case, in the container previously mentioned, with the villous surface of the placenta being oxygenated directly by placement over the Oxygenation Device; in the former case, the placenta can be placed with the villous surface adjacent to and touching the bottom portion of the net, the fetus then being placed thereon.

An alternative method would be to place the fetus in the net alone, and the placenta being placed over it on a Placental Dome made specifically for such a purpose and having an opening for continued, unpressured attachment of the umbilical cord to both the placenta and fetus. In this alternative method, oxygenation of the placenta would have to be performed by placement of the Oxygenation Device proximate to the villous surface and spraying bubbles directly through the fluid into the placental bed; or oxygenation of the placenta would have to be performed by previously described oxygenating vehicles being placed in the fluid, and thus flowing around and upon the placental villous surface.

It should be noted that in any method whereby the Placental Support dome is utilized, the pulse oximeter could be placed on the top of the dome, touching the placenta, to monitor placental oxygenation. Alternatively, if the placenta is of a shape and size consistent with remaining stable without folding upon itself, it could be placed in the net with the fetus resting upon it, with the placental villous surface next to the net.

Concerning the Most Ominous Case of Septic Delivery, a Vaginal Delivery Before Fetal Viability:

By passing through the vagina, the fetus, umbilical cord and placenta would be subjected to superficial bacterial contamination, and must therefore be washed as quickly and thoroughly as possible in antimicrobial agents. Again, the fetus must be kept from spontaneous respiratory attempts, placed in the above described container for oxidation and superficial decontamination, with successive washings in new containers of antibiotics and antimicrobials, either alone or in combination, removing as much mucus and superficial contamination as possible. Meanwhile, the placenta must be removed from the uterus as quickly as possible and given the treatment described above for septic delivery with ruptured membranes, as well as serial washing for removal of superficial contamination. Blood clots should be removed as thoroughly as possible from placental villous surface, without damaging the villous surface. The fetus, umbilical cord and placenta are then placed together and intact into the Conceptus Chamber with or without the Placental Dome, as described above.

Concerning Monitoring and Surveillance of the Conceptus:

A Best Method for surveillance of the health and well-being of the conceptus involves continuous and intermittent evaluations of the conceptus.

Continuous evaluation of the fetal heart rate by electronic, Doppler ultrasound is begun as soon as the conceptus is placed in the Conceptus Chamber. Continuous or periodic pulse oximetry of the conceptus is taken by a pulse oximeter in contact with the placenta. These could be monitored for evaluation by caretakers with permanent recordings, and settings placed to sound an alarm should either the fetal heart rate or oximeter readings be inconsistent with normal levels.

Monitoring of the constituents of the outgoing gas and the outgoing fluid may be performed continually or intermittently: these will aid in decisions toward any adjustment of the incoming gas and fluid constituents, as needed, to assure optimally balanced levels of all constituents required for fetal metabolism and growth. Monitoring of outgoing fluids could be made for pathogens.

Monitoring and evaluation of the developing conceptus can be performed outside the chamber by ultrasound and various radiographic techniques.

Concerning changes and Clinical Modification of the Constituents of the Fluid in the Chamber:

In addition to any unplanned changes indicated by evaluation of uptake of nutrients, or levels of metabolic by-products, of the conceptus, other changes may be planned and made commensurate with the normal changes in nutritional requirements as the fetus grows and matures.

Concerning a Conceptus Outgrowing the Conceptus Chamber:

Should a conceptus be delivered in a stage of development far from maturity, it may be delivered into a Conceptus Chamber suitable for its size but too small to accommodate the ultimate size required to complete fetal development. In such a case, when the conceptus has grown to the limits of the first chamber into which it was placed, it may be moved to a larger chamber by the following method:

First, the new chamber is prepared as previously described for initial use. Then, under sterile conditions and using standard sterile technique, the conceptus is transferred to the larger net by removing the smaller net from the smaller chamber, placing it into the net and fluid in the larger chamber, and separating the smaller net from its supporting ring. Thus the conceptus together with its initial supportive net are transferred intact, and remain intact, within the second chamber. This will prevent damage to the delicate villous structure of the placenta which has been growing in and around the threads of the supportive net.

Concerning Final Removal of the Conceptus from the Chamber:

The ultimate goal of the invention is to afford the fetus optimal development to the point of maturity. This point is determined by the practitioner. For example, in the human, since the final organ system to develop to viability is the lungs, they are tested for maturity by standard laboratory methods: one presently used method of determining fetal lung maturity is the ratio of lecithin to sphingomyelin in the amniotic fluid, which is performed in almost all maternity hospitals. When fetal lung maturity is found to be adequate, the Conceptus Chamber lid may be opened, the chorioamnion opened, and the baby delivered under normal standards of obstetrical care.

In cases involving other species, maturity will be determined by the practitioners and removal of the conceptus determined thereby.

Concerning Improvements Over Prior Art:

Certain embodiments of this invention constitute improvements over, and remove potential problems arising in, the prior art. The present invention provides support for the entire conceptus, that is, the fetoplacental unit with intact chorioamnion: this eliminates the need for maintaining a separate chamber and an amniotic fluid-like environment for the fetus, and a separate chamber and fluid system for the placenta; it also allows the chorioamnion and the amniotic fluid to continue performing their normal functions. The present invention eliminates the need for the central portion of the prior art Placental Chamber, which had been invented for the purpose of supporting the placenta separately and keeping two distinct fluid systems: the nutrient fluid separate and distinctly constituted from the lower chamber fluid, as described in the prior art Placental Chamber previously cited.

The method of use is simplified in many ways: there is a single chamber and top instead of two chambers and a separate midpiece; this minimizes surface areas and reduces steps necessary to place the fetoplacental unit in the Chamber. Further, it greatly simplifies the surgical method, since the placenta and fetus are not removed separately and placed in separate chambers while taking care not to harm the connecting umbilical cord. This also eliminates the complications and increased septic dangers of having to deliver the fetus and placenta separately, and to place them into the Chamber separately. There is a significant reduction in the time and skill needed to transfer the fetoplacental unit to the chamber at the time of delivery from the maternal womb.

There are no moving parts within the Conceptus Chamber, reducing danger of sepsis and obviating the possible catastrophic malfunction of moving parts within the Conceptus Chamber.

The present invention oxygenates the conceptus by introducing elemental oxygen, or oxygen-rich gases, under pressure into the fluid and around the conceptus directly, eliminating the need for oxygenating agents such as red bloods cells or other oxygen-carrying vehicles in the fluid. This is the first invention whereby a placenta or a conceptus is oxygenated directly by bubbles of elemental oxygen. This invention also describes a novel use of oxygenating agents such as ultrapure hemoglobin, mutant hemoglobin and fluorocarbons.

The Conceptus Chamber provides an open, supportive net to gently house the conceptus in a manner which will provide constant contact of fluid contents and oxygen to the entire surface area of the conceptus. By remaining in the natural state of being buoyantly immersed in fluid, the fetus is spared musculoskeletal and integumentary developmental problems encountered by severely premature infants in room air, which problems are caused by the pressure of their bodies on their resting surface, due to gravity. The net also provides gentle resistance to the fetal body movements, which is critical to normal musculoskeletal development of the fetus. The fluid may not need to be continually flowing in and out of the Chamber, reducing the possibility of contamination or danger of catastrophic pump malfunction.

Continuous monitoring of blood oxygenation and fetal heart rate are critical to provide early warning of anoxia. In U.S. Pat. No. 6,175,751, filed in March, 1999, Miazes teaches that there are two types of fetal pulse oximeters used to measure the oxygenation of a fetus: those touching presenting parts of the fetus, and those touching non-presenting parts; he describes another apparatus and method for sensing oxygen levels in a fetus. However, the present invention is the first to use pulse oximetry directly on the placenta to sense oxygen levels of the fetus, thereby assuring the highest level of accuracy, and avoiding the possibility of losing contact with the tissue being monitored by movement of the fetus or the development of vernix on the skin of the fetus.

Periodic evaluation of fetal growth parameters through the wall of the Conceptus Chamber by, for example, ultrasound can effectively direct indicated improvements in oxygen and/or nutrients supplied to the Conceptus Chamber. Monitoring of the outflow gases for carbon dioxide and outflow fluids for metabolic by-products such as urea could direct maintenance of healthy metabolism. Monitoring of the outflow fluid for bacteria or other contamination could be performed prophylactically, or during therapy to assess attainment of therapeutic goals in cases of sepsis or contamination.

The maintenance of the conceptus in the Conceptus Chamber could provide a simpler, more effective and more efficient method of caring for the most severely premature parturients than is available in the present state of the art in Neonatal Intensive Care Units. Such care and maintenance is notoriously labor-intensive, complicated and costly. Use of the Conceptus Chamber may result in reductions of both morbidity and mortality in appropriately selected cases, and for the first time will provide therapy for those cases which are too premature to have any hope of salvage utilizing the present state of the art.

New methods in technique described include treatment of a conceptus contaminated by a maternal intrauterine infection, or chorioamnionitis; and decontamination of a severely premature vaginal delivery; which have not been described heretofore.

Further changes and modifications may be made within the scope and concept of this invention.

In this invention I claim:

1. A chamber for sustaining the life of a conceptus; said chamber comprising
(a). lateral walls
which are connected to a closed bottom
which are partially or completely transparent for visualization of the interior
which provide a net opacity consistent with prevention of fetal damage secondary to light
comprised of material impervious to contamination of the interior
having support structures for maintaining a support structure for the conceptus
(b). a top opening, being covered by a removable lid
which lid can cover the chamber tightly enough to prevent contamination from outside air
which lid is equipped with a safety latch whereby it cannot accidentally be opened
(c). a support structure for the conceptus
in the form of an open net or basket type container
providing means of support for the conceptus within the fluid of the chamber
providing openings adequate for passage of gas and fluid to the conceptus within
comprising materials with flexibility adequate to allow normal fetal movement
said materials with rigidity adequate to provide normal resistance to fetal movement
having itself a rigid support for maintaining itself in position in the chamber
said rigid support fitting upon the side wall supports described for such purpose
said rigid support having handles for handling and moving the conceptus support
(d). inlet and outlet openings to the interior of the chamber for movement of gas
whereby direct oxygenation of the conceptus may be performed with elemental oxygen
whereby differences in the constituency of the incoming and outgoing gases provide indirect physiological surveillance of the conceptus which may direct changes in care
(e). inlet and outlet openings to the interior of the chamber for placement, removal, and movement of fluid
(f). devices on or within the chamber for direct fetal surveillance, comprising
a Doppler ultrasound transducer for continuous fetal heart rate monitoring
a pulse oximeter for evaluation of conceptus oxygenation status
which pulse oximeter can be applied to the placental surface of the conceptus
programmable alarm systems to alert staff of abnormalities in heart rate or oxygenation
(g). wherein all or part of the components of the chamber, including those here listed
comprise material containing antibiotic or antimicrobial substances, or both
comprise a coating of antibiotic or antimicrobial substances, or both
(h). a temperature control device
with programmable homeostatic temperature range
with programmable alarm system to alert staff of deviation from programmed temperature
(i). whereby said chamber provides a novel means of supporting a conceptus outside of the maternal womb
(j). whereby said chamber provides a novel use for materials containing antibiotic or antimicrobial substances, or both
(k). whereby said chamber provides a novel use for materials coated by antibiotic or antimicrobial substances, or both
(l). whereby said chamber provides a novel means of fetal surveillance
comprising pulse oxygenation reading of the conceptus via contact with the placenta
(m). whereby said chamber provides a novel means of oxygenation of a conceptus, by direct presentation of elemental oxygen to the placental villous surface.

2. A Fluid to be used within the Chamber of claim 1, comprising
(a). an aqueous solution and suspension of physiologically balanced salts and nutrients
(b). which fluid constituents can be compounded specifically for the needs of the conceptus
which constituents can be changed with the changing needs of the growing conceptus
and which fluid constitutes a novel means and method of nutrition of a conceptus
(c). which fluid constituents can be changed as indicated by surveillance of specific fluid constituents, whereby differences in the constituency of the incoming and outgoing fluids provide indirect nutritional and physiologic surveillance of the conceptus and its by-products of metabolism, which may direct changes in care
which comparison of differences constitutes a novel means of physiological and metabolical surveillance of a fetus or conceptus
(d). containing prophylactic or therapeutic antimicrobial agents or antibiotics, or both, when clinically indicated
which constitutes a new method of use of such agents
which constitutes a new method of prophylactic or therapeutic antimicrobial or antibiotic therapy
(e). containing oxygen transporting vehicles for oxygenation of the conceptus, when clinically indicated
said vehicles comprising ultrapure hemoglobin or mutant hemoglobins or fluorocarbons or a combination thereof
which constitutes a novel use of these oxygen transporting vehicles.

3. A Method of Oxygenation of the Chamber of claim 1, comprising
(a). an inlet for oxygen or oxygen-rich gas
which gas is transported through a hose or pipe to an oxygenating device at a controllable rate and pressure
(b). said oxygenating device being placed securely within the chamber
(c). said oxygenating device consisting of a closed chamber in which the gas enters through the said hose or pipe, and exits through holes in the wall of the oxygenating device
(d). said holes being of a number, placement and diameter consistent with optimal bubbling of the gas into the fluid of the chamber and upon the conceptus
(e). said oxygenation device providing a novel means of oxygenating a conceptus.

4. A Surgical Method utilized with the Chamber of claim 1, comprising
(a). pre-operative preparation, comprising
i. pre-operative assessment of the gestation
ii. pre-operative preparation of the chamber
iii. pre-operative preparation of the operating field
(b.) intraoperative methods of uterine entry, conceptus dissection and conceptus transfer
from the uterus to the chamber
which constitute a novel means of obstetrical delivery, whereby the conceptus remains intact and alive
i. in cases free of sepsis and with intact conceptus
ii. in cases with suspected or known sepsis or chorioamnionitis
whereby the conceptus is bathed and oxygenated in a physiologic solution
containing antimicrobial agents or antibiotics or both
which constitutes a novel method of treatment
and which constitutes a new use for the oxygenating device described in claim 3
iii. in cases with accidental cutting of the chorioamnion
whereby a novel method of surgical repair of the chorioamnion is described
iv. in cases of irreparable rupture of the chorioamnion
whereby a novel method of delivery of the conceptus is described
in which the fetus is prevented from respiring
in which the placenta, umbilical cord and fetus are placed separately into the chamber while remaining attached
v. in cases of vaginal delivery
whereby a novel method of delivery is described
in which the fetus is not allowed to respire
in which the placenta, umbilical cord and fetus are washed and disinfected as
thoroughly as possible, as described in cases of sepsis above, before placement in the chamber while still attached
whereafter antibiotics or antimicrobial agents or both are administered through the fluid of the chamber
vi. in cases of multiple gestation
(c). post-operative transfer of the chamber and enclosed conceptus from the operative field utilizing the dedicated cart of claim 6.

5. A method of performing surgery on a fetus or conceptus
whereby the placenta remains oxygenated by the said oxygenation device throughout the operation, such a placental oxygenation device being employed continuously while the surgical procedure is carried out, and the conceptus being placed in the chamber at the conclusion of surgery
which provides a new method of fetal surgery.

6. A dedicated, self-contained cart with easily sterilized surfaces for the chamber of claim 1
(a). of dimensions and stainless steel construction consistent with secure support of the chamber of claim 1
(b). securely supporting auxiliary apparatus, including
i. top and bottom shelves, and a utility shelf
ii. a gas tank with pressure and flow controls and monitors and alarm system
iii. a monitor for the fetal heart rate transducer with alarm system
iv. a monitor for the pulse oximeter with alarm system
v. a pump for the administration of fluid into the chamber, for the circulation of fluid within the chamber, and for the changing of the fluid, with alarm system
vi. a battery for actual or back-up supply of electrical energy
(c). transportable via locking wheels of a dimension large enough to minimize jarring
(d). incorporating monitors and alarm systems
for the fetal heart rate transducer of claim 1
for the pulse oximeter of claim 1
(e). which cart may be modified to be used in an ambulance, or in agricultural uses on a truck, or in the field, or in remote areas.

7. A method of utilizing the chamber of claim 1 for nonhuman species
(a). including other placental species, in which the chamber of claim 1, the dimensions of the net of claim 1 and the fluid of claim 2 would be modified to suit the particular species and gestational parameters of each case
(b). including nonplacental species, in which the chamber of claim 1, the dimensions and characteristics of the net or basket and the fluid of claim 2 would be modified to suit the particular species and gestational parameters of each case.
